(19) United States
(12) Patent Application Publication
(10) Pub. No.: US 2018/0374159 A1
(43) Pub. Date: Dec. 27, 2018

(54) HEALTH CARE MEDICAL CLAIMS COMPLETE PAYMENT SYSTEM FOR MULTIPLE PAYMENT PROCESSORS

(71) Applicant: ELECTRONIC COMMERCE FOR HEALTHCARE ORGANIZATIONS, INC., Westlake, OH (US)

(72) Inventors: William H. Davis, Fairview Park, OH (US); Ryan J. Davis, North Ridgeville, OH (US); Katharine A. Hoosenally, Westlake, OH (US); John J. Lombardi, Rocky River, OH (US); Kristopher Todd Kern, Strongsville, OH (US); Timothy Scott Adkins, Sequim, WA (US); Charles E. Kirkpatrick, Spring, TX (US); Tod David Logterman, Fairview Park, OH (US)

(73) Assignee: ELECTRONIC COMMERCE FOR HEALTHCARE ORGANIZATIONS, INC., Westlake, OH (US)

(21) Appl. No.: 15/962,777

(22) Filed: Apr. 25, 2018

Related U.S. Application Data
(60) Provisional application No. 62/489,797, filed on Apr. 25, 2017.

(51) Int. Cl. G06Q 40/08; G16H 10/60

(52) U.S. Cl. CPC G06Q 40/08; G16H 40/20; G16H 10/60

(57) ABSTRACT

Disclosed is a system and method of providing a complete payment of a medical claim to a medical service provider. According to an exemplary embodiment, multiple insurer/employer payer processers each provide funds associated with an insurer's/employer's portion of a claim and funds associated with the patient's portion of the claim to a central financial interchange settlement process which provides a complete payment to the medical service provider. According to an exemplary embodiment, the financial interchange is operatively associated with a patient reserve trust which operates as a guarantor of the patient portion funds paid to the medical service provider in the event the patient's adjudicated portion is ultimately unpaid by the patient.

This application claims the priority benefit of U.S. Provisional Patent Application Ser. No. 62/489,797, filed Apr. 25, 2017, by William H. Davis, and entitled “MULTIPLE SOURCE HEALTH CARE PAYMENT SYSTEM” and is incorporated herein by reference in its entirety.

### BACKGROUND

The present invention pertains to the payment of insurance claims, particularly medical insurance claims.

Currently, there are thousands of medical health insurance plans. Major employers negotiate custom medical insurance plans for their employees. Other companies select one of several insurance plans offered by an insurance company which may or may not include various options. Small business associations negotiate yet other health insurance contracts. The employees within these various employer groups obtain medical services at a plurality of covered medical facilities. Conversely, the various medical facilities treat patients with a myriad of different health plans.

The Employee Retirement Income Security Act (ERISA) prohibits the co-mingling of health insurance payments. A single instrument, such as a check, cannot be issued that shares risk. This results in insurance companies issuing a very large number of checks, and medical providers receiving large numbers of checks. Fulfillment vendors function as a go-between between the payers and the medical service providers. However, they too are bound by the ERISA rules against co-mingling of funds and must take care to avoid co-mingling funds received from payers and print or produce a multitude of checks to the various medical providers.

The funds are accompanied by an Explanation of Benefits (EOB) which is formatted and the contents normalized to the ANSI-835 standard. However, the normalized content is not standardized from employer group to employer group. Even though the normalized content may specify such terms as “non-covered” or “pending”, different health insurance contracts give different meanings to these terms. For example, a “non-covered” service in from one contract can mean that the service provider must write-off the amount while in another contract the insured is responsible for payment of the “non-covered” amount. Secondary insurers add other possible interpretations of “non-covered”. It is often difficult for the medical provider to determine, to a certainty, such simple information as the patient's co-pay, what amounts may be billed to the patient, which amounts must be written off, and the like. Determining this information generally requires a custom interpretation of the EOB from each of the various employer groups. Such individual interpretation is labor-intensive and expensive to the medical service providers. Moreover, due to the uncertainty regarding which charges must be written off and which may be billed to the patient, patients are often billed for charges which their insurance contract requires to be written off.

Only certain financial instruments meet ERISA regulations against co-mingling of funds. Certain legal entities such as trusts may be required for use of certain financial instruments. In addition, not all payees accept all types of financial instruments. Some financial instruments require electronic methods and access to certain networks and/or servers. Furthermore the financial instrument used to move funds and the EOB are intrinsically linked. A particular financial instrument may require a specific type of format for transmission of associated EOBs. As new financial instruments are developed and/or options for EOB formats permitted, system complexity for incorporation of new payment methods grows quickly.

Currently there are many health care medical payment processor companies who process adjudicated medical claims for insurers and employers. These medical payment processor companies (i.e., Payer Processors) provide payments to medical service providers. One example of a payer processor service is ECHO® which provides a consolidated financial instrument to medical service providers using an intelligent router to provide a preferred method of payment to the medical service provider as described in U.S. Pat. No. 8,626,536 by Davis et al., issued Jan. 7, 2014, and entitled “INTELLIGENT ROUTER FOR MEDICAL PAYMENTS”. The intelligent router provides a payment process which accommodates a plurality of payment protocols, formats, etc.

To provide a complete payment to a medical service provider for a specific medical claim, the ECHO payer process includes Symplicity® technology to combine an insurer's payment portion and a members/patient's portion associated with a deductible, etc. to provide a “complete payment” to a medical service provider. This “complete payment” includes the total amount of the adjudicated claim (insurer's portion+patient's portion) minus a discount, for example 5-15%. While the medical service provider receives a total payment less than the total of the adjudicated claim, the medical service provider receives the benefit of a timely complete payment and avoids the expense of billing and collecting the patient portion from the patients. It is common practice for the insurer payer processors to provide to a medical service provider only payment of the insurer's portion of an adjudicated claim. This disclosure and the exemplary embodiments disclosed herein provide a system and process to provide a complete payment to a medical service provider from multiple medical claims payer processors. Central to the disclosed complete payment system/process is a Financial Interchange Settlement and Patient Reserve trust which processes complete payments to medical service providers from multiple payer processors.

### INCORPORATION BY REFERENCE

U.S. Pat. No. 4,858,121, issued Aug. 15, 1989, by Barber et al. and entitled “MEDICAL PAYMENT SYSTEM”;

U.S. Pat. No. 8,626,536, issued Jan. 7, 2014, by Davis et al. and entitled “INTELLIGENT ROUTER FOR MEDICAL PAYMENTS”;

U.S. Patent Publication No. 2014/0088999, published Mar. 27, 2014, by Davis et al. and entitled “MEDICAL CLAIMS PAYMENT SYSTEM WITH PAYMENT CONSOLIDATION FROM MULTIPLE EMPLOYER ACCOUNTS”; and

U.S. Patent Publication No. 2014/0095195, published Apr. 3, 2014, by Davis et al. and entitled “INTELLIGENT ROUTER FOR MEDICAL PAYMENTS”, are incorporated herein by reference in their entirety.

### BRIEF DESCRIPTION

In one embodiment of this disclosure, described is a computer implemented method of providing a complete payment of an adjudicated medical claim including an insurer portion and an associated patient portion to a medical service provider comprising: using one or more processors: a) a complete payment financial interchange system operatively associated with a plurality of health insurance payment processing systems electronically receiving an insurer's data, insurer's portion of funds and patient's portion of funds associated with an adjudicated medical insurance claim for a medical service provider from one of the plurality of health insurance payment processing systems; b) the complete payment interchange system electronically receiving an insured patient's data insurer's portion of funds, and patient's portion of funds, and to electronically transfer a single complete payment to the medical service provider, the single complete payment including a discounted payment amount relative to a total amount associated with the adjudicated medical insurance claim; and c) the complete payment interchange system funding a patient reserve trust to guarantee the patient's portion of the adjudicated medical insurance claim.

In another embodiment of this disclosure, described is a medical claims complete payment interchange system comprising: a plurality of health insurance payment processing systems configured to electronically receive an insurer's data, an insurer's portion of funds and a patient's portion of funds associated with an adjudicated medical insurance claim for a medical service provider; a complete payment interchange system operatively associated with the plurality of health insurance payment processing systems, the complete payment interchange system configured to: a) electronically receive from one of the plurality of health insurance payment processing systems an insurer's data, an insurer's portion of funds and patient's portion of funds associated with a first adjudicated medical insurance claim for a medical service provider medical insurance claim; b) electronically transfer a single complete payment to the medical service provider, the single complete payment including a discounted payment amount relative to a total amount associated with the adjudicated medical insurance claim; and c) the complete payment interchange system funding a patient reserve trust with an amount of funds associated with the discounted payment amount to guarantee a patient funding.

### BRIEF DESCRIPTION OF THE DRAWINGS

FIG. 1 is a flow chart of a health care medical claims complete payment system process for multiple payment processors according to an exemplary embodiment of this disclosure;

FIG. 2 is a flow chart of a complete payment system process according to an exemplary embodiment of this disclosure, the complete payment system including a patient reserve trust;

FIG. 3 is a flow chart of another complete payment system process according to an exemplary embodiment of this disclosure, the complete payment system including a patient reserve trust and a receivables management process;

FIG. 4 is a flow chart of a complete payment system eligibility process according to an exemplary embodiment of this disclosure;

FIG. 5 is a flow chart of a complete payment system financial transaction process according to an exemplary embodiment of this disclosure;

FIG. 6 is another flow chart of a complete payment system according to an exemplary embodiment of this disclosure; and

FIG. 7 is a flow chart of a complete payment system according to an exemplary embodiment of this disclosure illustrating benefits of efficiencies, liquidity and clarity.

### DETAILED DESCRIPTION

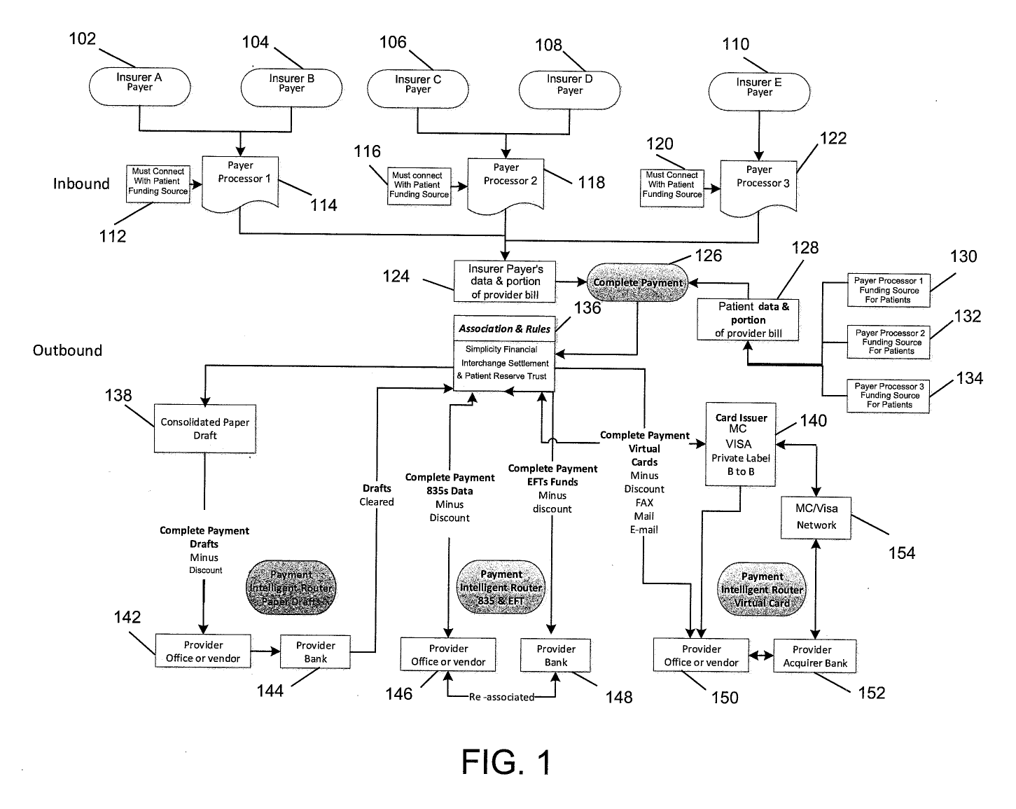

With reference to FIG. 1, disclosed is a flow chart of a health care medical claims complete payment system process for multiple payment processors according to an exemplary embodiment of this disclosure.

The Simplicity Financial Interchange Service charges its payer members a fee in exchange for eliminating its provider fulfillment transactional costs and the costs to support new mandated provider payment options. The disclosed simplicity Financial Interchange will deliver consolidated financial transactions to medical providers by paper for free or, at the provider's option, provide eft payments and adjudicated data via various payment networks charging various fees. Thus, following the proven successful business models of other financial services.

The simplicity Financial Interchange Service will aggregate smaller payers into a standard settlement process that medical providers may use to process these payer payments as efficiently as larger payer customers by providing small payers the payment efficiencies of their largest competitors by sharing the costs of these processes across an association of payers rather than incurring these costs alone.

Payers must be sponsored by a payor processor who is a member of the association, i.e., SPA (Simplicity Payment Association). They will be required to provide the information necessary for the processor to properly collect and submit the information and funds to meet the association's requirements to process each provider transaction.

Processors will sponsor each payer in SPA and will be responsible for collecting and funding the transactions from payers and placing them into the association. They will train payers to use the association's settlement service processes, provide customer service for their payers for cards issued such as opt out, assist payers in meeting the association's requirement for data exchange and print any paper consolidated provider documents.

The Association has established rules under which the payors will exchange their data and dollars with providers and processors will be on the Association board to regulate the rules that have been established. The Association establishes the net Simplicity Financial Interchange fees and defines the type of services the Association will develop for its members based on recommendation from payer processors and their own settlement services.

The Simplicity Financial Interchange Settlement and Trust provides the delivery and settlement services of all its payer members to medical service providers based on the rules established by the Association. It also assists its payor processors with training and enrollment of it payer's members, settlement and data issues with providers as well as purchasing or providing services to deliver the services approved by the association.

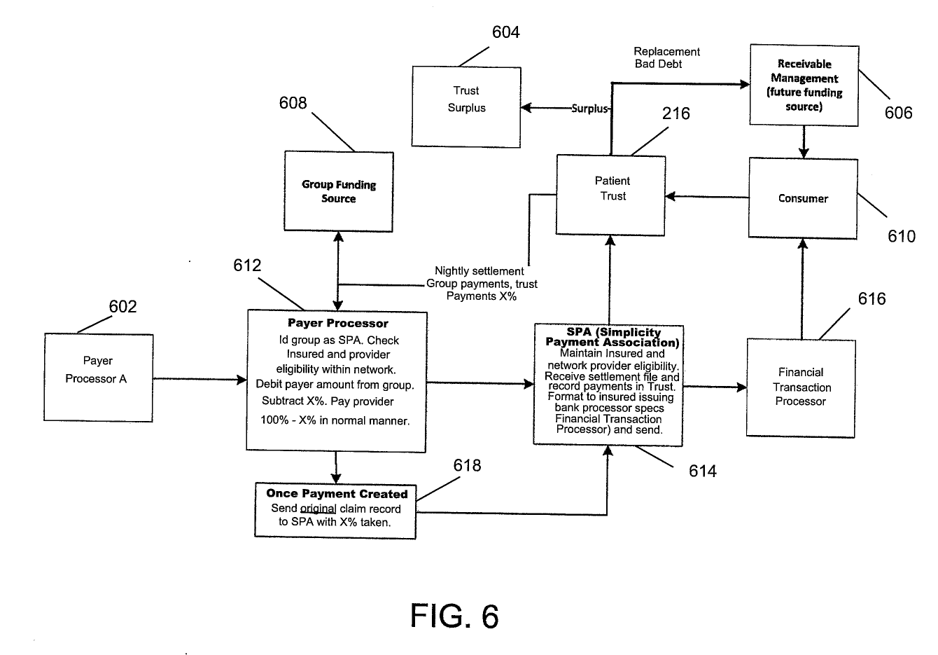

As shown in FIG. 6, payer processor identifies SPA (Simplicity Payment Association) groups and associated PPO (Preferred Provider Organizations) and sends data for all in-network services to SPA and will not send 835 to provider. In addition, payer processor approves payments, and supplies SPA current eligibility including PPO (Preferred Provider Organization) name, group name and address deductible co-ins.

SPA debits funding account verifies that TIN (Tax ID No.) is SPA and verifies Insured's status with Patient Reserve Trust is active. If both verify as yes, then SPA processes the transaction.

The SPA transaction funds patient obligations minus co-payment & NC (Not Covered) and subtracts a discount percentage, for example 7%, (current reserve) from both amounts and processes as a single complete payment to a medical service provider in the provider's preferred payment method.

SPA then sends payment information to the issuer payer's funding source identified with relevant group.

Result: (Complete payment) full funded payment to provider with zero patient obligation placed to issuer

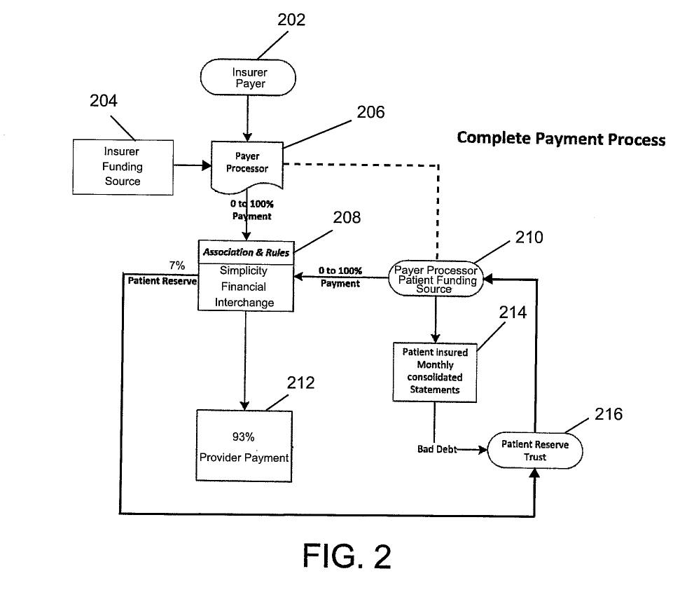

With reference to FIG. 2, disclosed is a flow chart of a complete payment system process according to an exemplary embodiment of this disclosure, the complete payment system including a patient reserve trust.

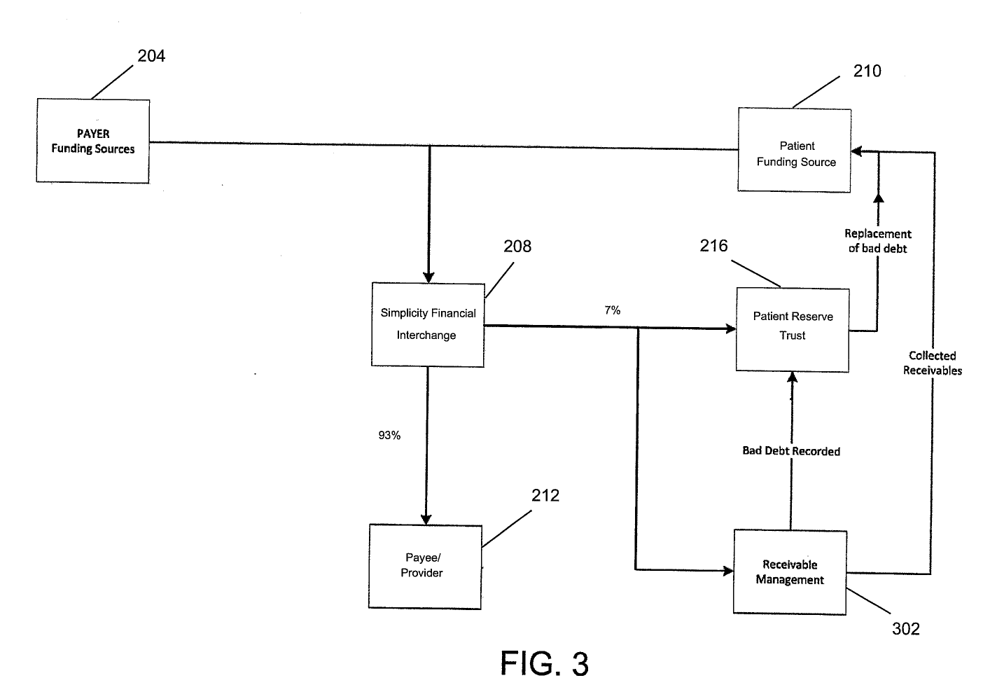

With reference to FIG. 3, disclosed is a flow chart of another complete payment system process according to an exemplary embodiment of this disclosure, the complete payment system including a patient reserve trust and a receivables management process.

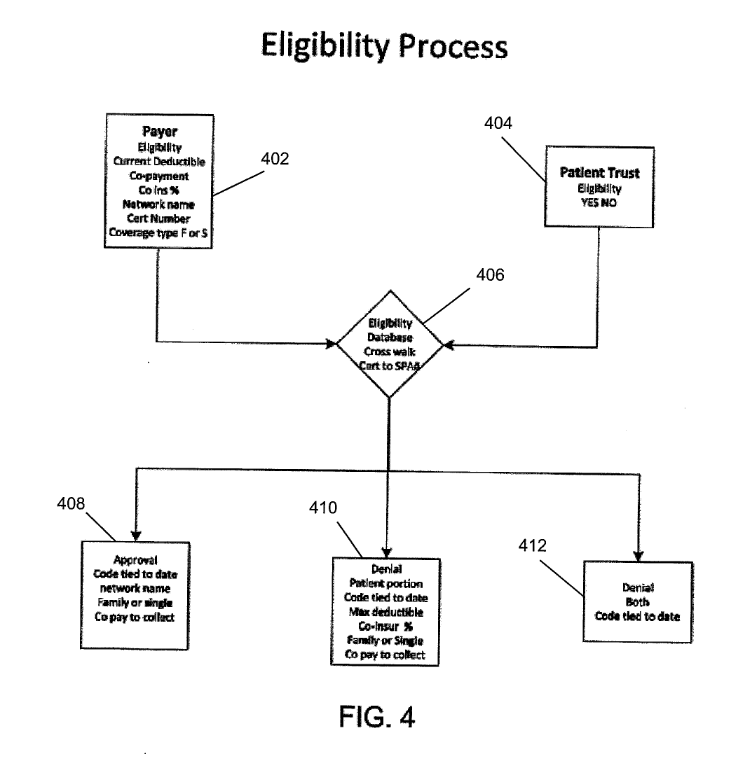

With reference to FIG. 4, disclosed is a flow chart of a complete payment system eligibility process according to an exemplary embodiment of this disclosure.

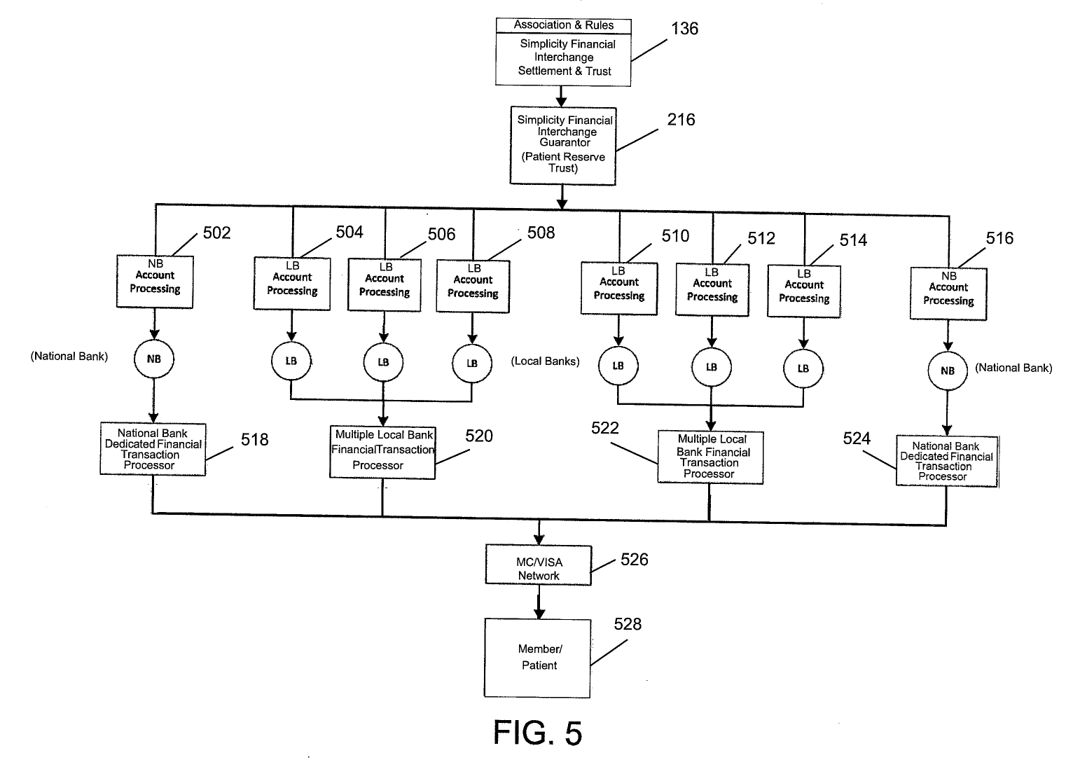

With reference to FIG. 5, disclosed is a flow chart of a complete payment system financial transaction process according to an exemplary embodiment of this disclosure.

With reference to FIG. 6, disclosed is another flow chart of a complete payment system according to an exemplary embodiment of this disclosure.

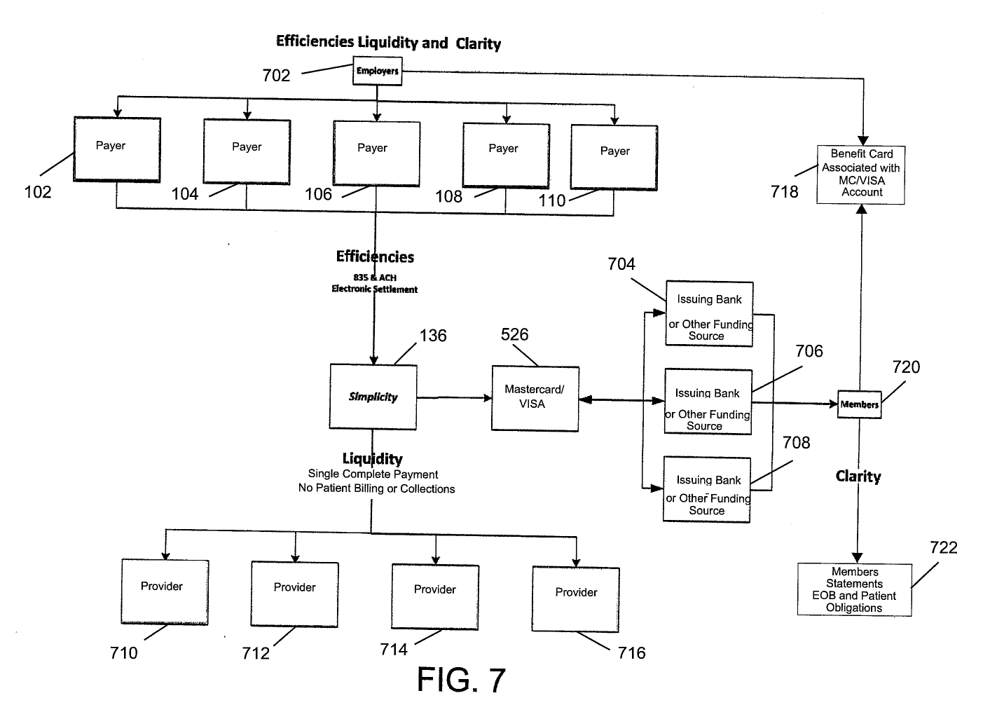

With reference to FIG. 7, disclosed is a flow chart of a complete payment system according to an exemplary embodiment of this disclosure illustrating benefits of efficiencies, liquidity and clarity.

Reference Character Description Table

| Reference | |
|---|---|
| Character | Description |
| 102 | Insurer A Payer |
| 104 | Insurer B Payer |
| 106 | Insurer C Payer |
| 108 | Insurer D Payer |
| 110 | Insurer E Payer |
| 112 | Patient Funding Source |
| 114 | Payer Processor 1 |
| 116 | Patient Funding Source |
| 118 | Payer Processor 2 |
| 120 | Patient Funding Source |
| 122 | Payer Processor 3 |
| 124 | Insurer Payer's data and portion of provider bill |
| 126 | Complete Payment |
| 128 | Patient data and portion of provider bill |
| 130 | Payer Processor 1 Funding Source for Patients |
| 132 | Payer Processor 2 Funding Source for Patients |
| 134 | Payer Processor 3 Funding Source for Patients |
| 136 | Simplicity Financial Interchange |
| 138 | Consolidated Paper Draft |
| 140 | Card Issuer |
| 142 | Provider Office or Vendor |
| 144 | Provider Bank |
| 146 | Provider Office or Vendor |
| 148 | Provider Bank |
| 150 | Provider Office or Vendor |
| 152 | Provider Acquirer Bank |
| 202 | Insurer Payer |
| 204 | Insurer Funding Source |
| 206 | Payer Processor |
| 208 | Simplicity Financial Interchange |
| 210 | Payer Processor Patient Funding Source |
| 212 | Provider Payment |
| 214 | Patient Monthly Consolidated Statements |
| 216 | Patient Reserve Trust |
| 302 | Receivable Management |
| 402 | Payer Eligibility |
| 404 | Patient Trust Eligibility |
| 406 | Eligibility Database |
| 408 | Approval Code Generation |
| 410 | Denial Code Generation |
| 412 | Denial Code Generation |
| 502 | NB Account Processing |
| 504 | LB Account Processing |
| 506 | LB Account Processing |
| 508 | LB Account Processing |
| 510 | LB Account Processing |
| 512 | LB Account Processing |
| 514 | LB Account Processing |
| 516 | NB Account Processing |
| 518 | National Bank Dedicated Financial Transaction Processor |
| 520 | Multiple Local Bank Financial Transaction Processor |
| 522 | Multiple Local Bank Financial Transaction Processor |
| 524 | National Bank Dedicated Financial Transaction Processor |
| 526 | MC/VISA Network |
| 528 | Member/Patient |
| 602 | Payer Processor A |
| 604 | Trust Surplus |
| 606 | Receivable Management (future funding source) |
| 608 | Group Funding Source |
| 610 | Consumer |
| 612 | Payer Processor Process |
| 614 | SPA Process |
| 616 | Financial Transaction Processor |
| 618 | Send original claim record to SPA |
| 702 | Employers |
| 704 | Issuing bank or other funding source |
| 706 | Issuing bank or other funding source |
| 708 | Issuing bank or other funding source |
| 710 | Provider |
| 712 | Provider |
| 714 | Provider |
| 716 | Provider |
| 718 | Benefit Card Associated with MC/VISA Account |
| 720 | members |
| 722 | Members Statements EOB and Patient Obligations |

The exemplary embodiment has been described with reference to the preferred embodiments. Obviously, modifications and alterations will occur to others upon reading and understanding the preceding detailed description. It is intended that the exemplary embodiment be construed as including all such modifications and alterations insofar as they come within the scope of the appended claims or the equivalents thereof.

1. A computer implemented method of providing a complete payment of an adjudicated medical claim including an insurer portion and an associated patient portion to a medical service provider comprising:
using one or more processors:
a) a complete payment financial interchange system operatively associated with a plurality of health insurance payment processing systems electronically receiving an insurer's data, insurer's portion of funds and patient's portion of funds associated with an adjudicated medical insurance claim for a medical service provider from one of the plurality of health insurance payment processing systems;
b) the complete payment interchange system electronically receiving an insured patient's data insurer's portion of funds, and patient's portion of funds, and to electronically transfer a single complete payment to the medical service provider, the single complete payment including a discounted payment amount relative to a total amount associated with the adjudicated medical insurance claim; and
c) the complete payment interchange system funding a patient reserve trust to guarantee the patient's portion of the adjudicated medical insurance claim.

2. A medical claims complete payment interchange system comprising:
a plurality of health insurance payment processing systems configured to electronically receive an insurer's data, an insurer's portion of funds and a patient's portion of funds associated with an adjudicated medical insurance claim for a medical service provider;
a complete payment interchange system operatively associated with the plurality of health insurance payment processing systems, the complete payment interchange system configured to:
a) electronically receive from one of the plurality of health insurance payment processing systems an insurer's data, an insurer's portion of funds and patient's portion of funds associated with a first adjudicated medical insurance claim for a medical service provider medical insurance claim;
b) electronically transfer a single complete payment to the medical service provider, the single complete payment including a discounted payment amount relative to a total amount associated with the adjudicated medical insurance claim; and
c) the complete payment interchange system funding a patient reserve trust with an amount of funds associated with the discounted payment amount to guarantee a patient funding.
